(19) United States
(12) Plant Patent Application Publication
(10) Pub. No.: US 2011/0145956 P1
(43) Pub. Date: Jun. 16, 2011

(54) Apple Tree Named 'New York 1'

(50) ;

(76) Inventors: Susan K. Brown, Geneva, NY (US); Kevin Maloney, Phelps, NY (US)

(21) Appl. No.: 12/653,569

(22) Filed: Dec. 15, 2009

(51) Int. Cl. A01H 5/00

(52) U.S. Cl. PLT/172

(57) ABSTRACT

A new and distinct Malus domestica apple tree variety named ‘New York 1,’ particularly characterized by having attractive red fruits that are sweet, juicy and have a crisp texture and ripen early mid-season. The fruit of ‘New York 1’ has excellent quality at harvest that it maintains during long storage (100-150 days). Trees are annually productive. ‘New York 1’ was tested as NY 98804-001.

### 1. LATIN NAME OF THE GENUS AND SPECIES OF THE PLANT CLAIMED

Malus domestica

### 2. VARIETY DENOMINATION

New York 1

### 3. BACKGROUND OF THE INVENTION

The present invention relates to a new and distinct variety of apple tree botanically known as Malus domestica ‘New York 1’ and hereinafter referred to by the variety denomination of ‘New York 1’.

‘New York 1’ was selected for its precocious fruiting, attractive fruits and excellent fruit quality at harvest and after cold storage. ‘New York 1’ fruit are very crisp, juicy and sweet.

The new variety was derived from a controlled pollination in 1998 between the apple variety ‘Honeycrisp’ (U.S. Plant Pat. No. 7,197) and the apple breeding selection NY 752. NY 752 is a hybrid of ‘Starkspur Golden Delicious’×NY 88 (‘Monroe’×‘Melrose’). One seedling, designated NY 98804-001, was selected from a population of 381 seedlings on the basis of its attractive fruits, precocity and excellent fruit quality. Pollination, cultivation and selection were conducted at the New York State Agricultural Experiment Station Research South Farm in Geneva, N.Y. Additional trees of this seedling were produced by clonal propagation in 2002 and subsequent years afterward at the New York State Agricultural Experiment Station Research South Farm in Geneva, N.Y.

NY 98804-001 is being named and released as ‘New York 1’ and is the subject of this invention.

Asexual reproduction by budding of the new cultivar ‘New York 1’ by the inventors shows that the unique combination of characteristics of asexually propagated trees is true to form and transmitted through succeeding propagations.

### 4. BRIEF SUMMARY OF THE INVENTION

The following traits of the new variety have been observed and documented in multiple years. The observations and description were collected from test trees grown in Geneva, N.Y. and other test sites within New York State.

The new variety is distinguishable from its parent varieties ‘Honeycrisp’ and NY 752. Multiple features distinguish the new variety from the parent varieties.

‘New York 1’ has similar characteristics to ‘Honeycrisp’ in its sweetness, juiciness and crisp texture, but it is not prone to bitter pit, biennial bearing and other production challenges of ‘Honeycrisp’ such as susceptibility to soft scald. ‘Honeycrisp’ leaves have a distinctive leaf mottling disorder that is not seen on ‘New York 1’.

When compared to ‘New York 1,’ ‘Honeycrisp’ fruit have lighter and less uniform fruit color, globose fruit shape, and earlier maturity. NY 752 fruit has later fruit maturity, larger fruit size, less full red color, and a globose fruit shape when compared to ‘New York 1.’ NY 752 fruits are also prone to fruit russetting.

‘New York 1’ has smaller average fruit size than either parent and a darker and more uniform red color pattern and more extensive surface color of the fruit.

Fruit of ‘New York 1’ mature after ‘Honeycrisp’ and before NY 752. ‘New York 1’ is unique from either parent in having a conic and uniform fruit shape, sweeter flavor, and longer storage life.

‘New York 1’ has not been grown and observed in all geographic locations and possible climatic conditions. Slight phenotypic variations might be observed over locations without any change to the genotype.

### 5. BRIEF DESCRIPTION OF THE DRAWINGS

The accompanying color photographs illustrate typical specimens of the foliage and fruit and typical anatomical characteristics of the new Malus variety ‘New York 1,’ showing the colors as true as is reasonably possible with colored reproductions of this type. Colors in the photographs may differ slightly from the color values cited in the detailed botanical description, which accurately describe the color of ‘New York 1.’

FIG. 1 shows one flower cluster with the king flower open of the ‘New York 1’.

FIG. 2 shows 6 different flowers, two flowers each (from left to right) of the variety ‘Honeycrisp’ (far left), ‘New York 1’ (middle) and NY 752 (far right).

FIG. 3 shows six views of a representative apple of ‘New York 1’. From the upper left hand corner and proceeding clockwise:

View A is a cross-sectional view of ‘New York 1’ fruit cut longitudinally.

View B is a side view.

View C is a stem-end view.

View D is a bottom or calyx-end view.

View E is a side view.

View F is a cross-sectional view of ‘New York 1’ fruit cut horizontally.

FIG. 4 shows two mature leaves of ‘New York 1,’ one top view and one bottom view of a typical mature leaf.

FIG. 5 shows ‘New York 1’ apples on the tree.

FIG. 6 shows a row of young ‘New York 1’ trees.

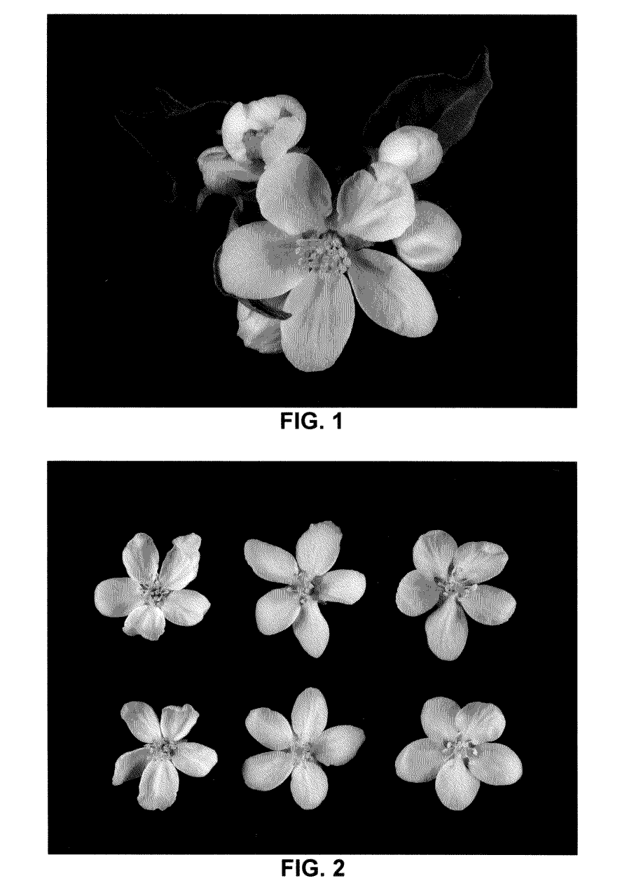

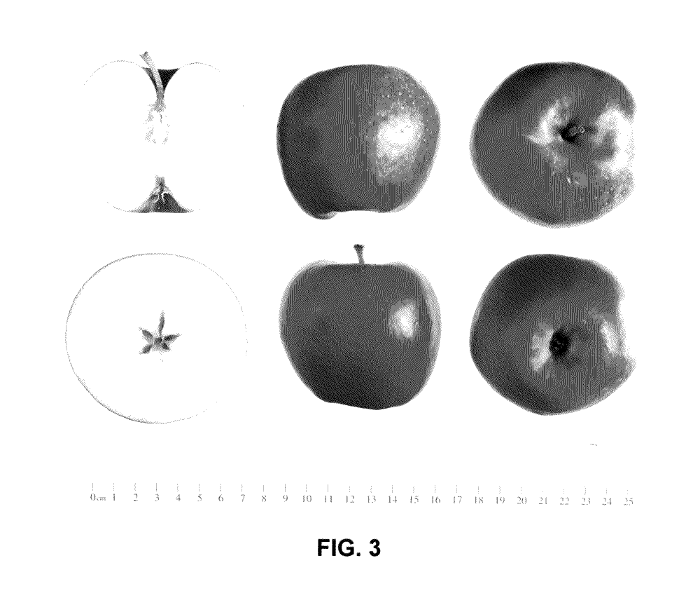

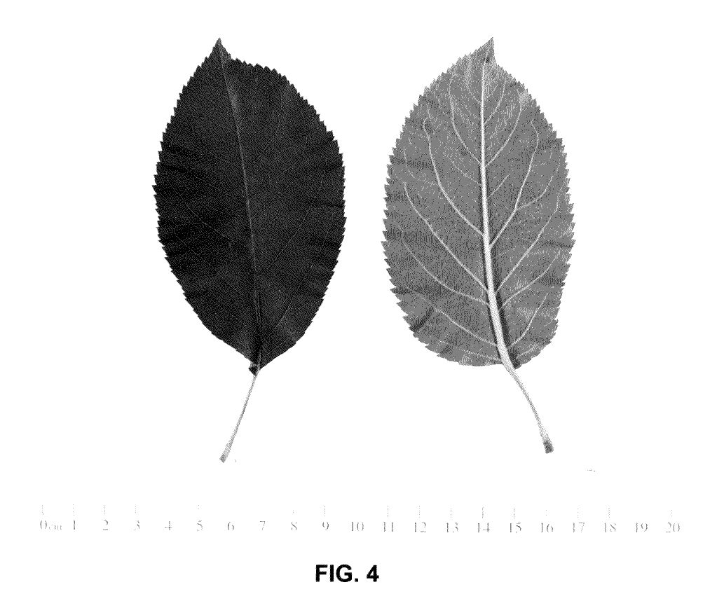

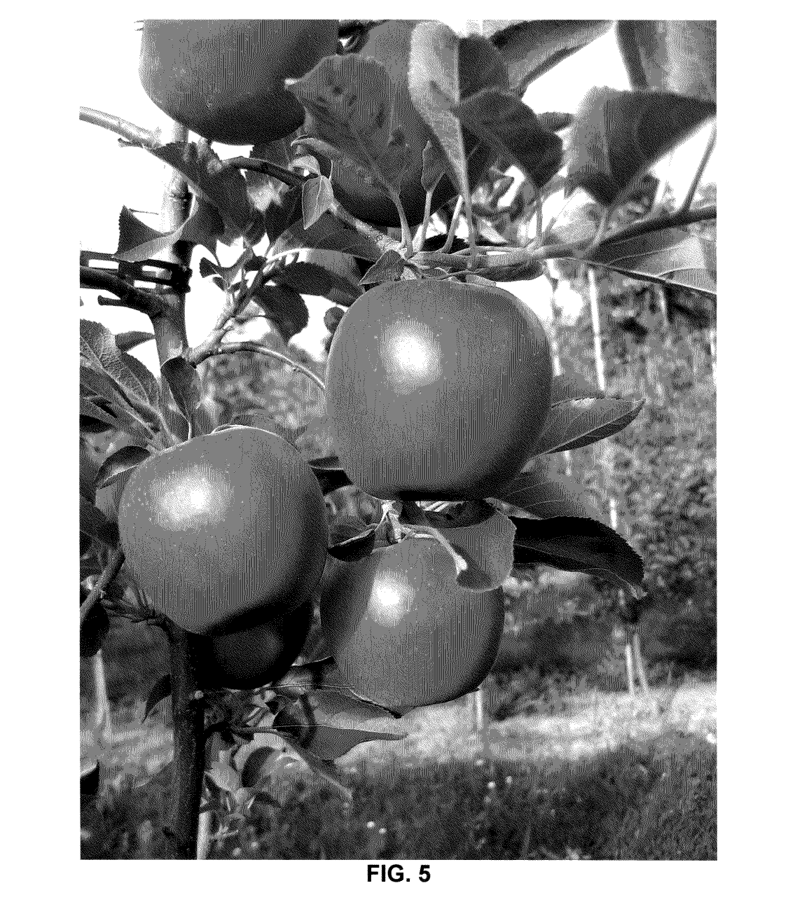

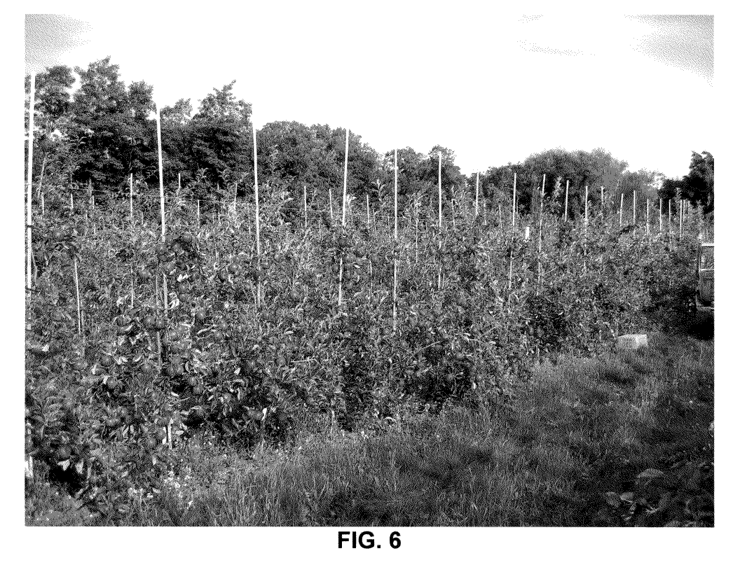

### 6. DETAILED BOTANICAL DESCRIPTION

The following is a detailed botanical description of the new variety with color terminology in accordance with The Royal Horticultural Society Colour Chart (R.H.S.C.C.) except where general color terms of ordinary meaning are used as is clear from the context.

The specimens described were grown at Cornell University's New York State Agricultural Experiment Station in Geneva, N.Y. in the United States of America. The observations were made from trees fruiting during 2002 through 2008.

Tree flowering commences on average by May 7th with trees in full bloom by May 12th in Geneva, N.Y. The fruit ripened for eating towards early mid-season. Specifically harvest commences on about September 20th and ends about October 2^{nd} in Geneva, N.Y.
Trees: Small to medium; upright habit; early bearing on spurs; vigor low.
Trunk: Smooth, size, medium; the one-year old bark is RHSCC greyed-orange 166B and grey-brown 199A.
Branches: Moderately thick; smooth; multi-branching; the angle of branching being commonly 45 degrees above the horizontal.
Lenticels: Average; small.
Leaves: Medium size, upward pose, concavo-convex in cross section, serrate indentation of margin, medium glossiness of upper sides; medium pubescence on lower side; medium petiole length; medium stipule size; medium time of bud burst. Average leaf blade length 102 mm, blade width 53 mm, petiole length 33 mm and averages 4.1 serrations per cm of leaf blade margin. The color of the upper surface of the leaf blade is most similar to RHCSS 137A, the bottom surface of the leaf blade is most similar to RHCSS 147B, the veins are 145C and the petioles are RHSCC 148D.
Flowers: Medium time of beginning of flowering (10% flowers); six flowers per cluster, flowers in cluster overlap, medium size (47 mm flower diameter); flat shape; margins of petals barely touching; bud color of unopened flower, light pink (RHSCC Red group 54A).
Flower petal color: white (RHSCC White group 155D), 5 petals per flower, petal shape-oval, petal length 21 mm, petal width 14 mm.
Flower pedicel color: RHSCC Yellow green 146B, length 20 mm, width 1 mm.
Stamens: 20 stamens per flower, 7-9 mm long, and stamen color RHSCC white group 155A. Anther color RHS greyed yellow 162B.
Stigma: 7 mm long, 5 styles per flower — style color greyed green 193A, stigma color RHSCC grey brown 199C.
Sepals: Sepal color RHSCC green group 138B, sepal length 7 mm, sepal width 3 mm, and sepals in reflexed position.
Fruit:
Fruit: Examined at peak maturity.
Soluble solids: 15.3 Brix.
Acidity: 0.41% malic acid as determined by titration.
Lbs. Pressure: 17-19 as measured with a penetrometer.
Size: Medium; axial diameter, 7.3 centimeters, transverse diameter 6.3 centimeters, weight 168 grams.
Shape: Uniform; medium to long conical; symmetrical in side view; medium crowning at distal end.
Cavity: Slightly acuminate. RHSCC yellow-green 152C and ranging from 152A to 152D. Depth 14.0 mm, Width 24.6 mm.
Basin: Deep, medium breadth; regular and smooth. Depth 14.8 mm, Width 24.4 mm.
Stem: Medium thickness (2.4 mm) and medium length (17.2 mm).
Calyx: Persistent, closed, erect.
Calyx tube: Cone shaped.
Sepal: Length, medium; spacing, touching.
Stamen remnants: Basal.
Core lines: Meeting.
Core position: Median, closed core, small core (20 mm in width).
Carpels: Elliptical, inner surface of carpel smooth.
Skin: Medium-thick; smooth; bloom of skin, present; cracking tendency of skin, absent; background color, RHSCC greyed-yellow 162B and 160B. Prominent lenticels.
Over-color: Approximately ninety percent of over-color of skin; red RHSCC 53B and red 53A, greyed-red180A; mostly solid blush.
Flesh: Juicy; firm (17-19 lbs. at harvest).
Texture: Crisp and firm.
Flavor: High Brix 15.3; 0.41% titratable acid. The flavor is sweet and mild with a light acid balance.
Weight of fruit: 168 gm.
Quality: Excellent, fresh, juicy and crisp.
Seeds: Five locules. 8 to 10 seeds total. Seed length 8.2 mm.; seed width 4.9 mm.; seed depth 2.6 mm.; acuminate; dried color RHSCC 165A.
Use: Market; dessert, fresh slice, multiple.
Keeping quality: Excellent keeping quality with no disorders after 100 days.
Resistance to insects: Good.
Resistance to diseases: Good.
Production: Early and regular cropping. Very precocious.
Growth habit: Standard habit, fruit borne on short spurs.
Management: Trees require pruning in winter and fruit thinning in early summer. Trees in test plot trained as center leader or slender spindle trees. Natural habit is weak, rounded crown (fruiting leader), with many branches. ‘New York 1’ has been tested on M.9 (337), ‘Pajam 2’ and EMLA 9 rootstocks.

1. A new and distinct Malus domestica apple tree variety named ‘New York 1’ as described and illustrated herein.
